(19) United States
(12) Patent Application Publication
(10) Pub. No.: US 2018/0354872 A1
(43) Pub. Date: Dec. 13, 2018

(54) METHOD FOR PRODUCING BUTADIENE BY OXIDATIVELY DEHYDROGENATING N-BUTENES

(71) Applicants: BASF SE, Ludwigshafen am Rhein (DE); Linde AG, Munich (DE)

(72) Inventors: Jan Pablo JOSCH, Neustadt (DE); Ragavendra Prasad BALEGEDDE RAMACHANDRAN, Limburgerhof (DE); Philipp GRUENE, Mannheim (DE); Steffan RISSEL, Kirchheim (DE); Martin GEMBALA, New Orleans, LA (US); Ulrike WENNING, Pullach (DE); Anton WELLENHOFER, Hohenschaeftlarn (DE); Christine TOEGEL, Neubiberg (DE); Hendrik REYNEKE, Munich (DE)

(21) Appl. No.: 15/780,725

(22) PCT Filed: Dec. 2, 2016
(86) PCT No.: PCT/EP2016/079534

§ 371 Date: Jun. 1, 2018

(30) Foreign Application Priority Data
Dec. 3, 2015 (EP) 15197757.6

(51) Int. Cl. C07C 5/48; C07C 7/00; C07C 7/08; C07C 7/09; C07C 7/11; C07C 11/167; B01D 3/40; B01J 19/00

(52) U.S. Cl. CPC C07C 5/48; C07C 7/005; C07C 7/08; C07C 7/09; B01J 2219/00121; C07C 11/167; B01D 3/40; B01J 19/0053; B01J 2219/00425; C07C 7/11

(57) ABSTRACT

A) provision of a feed gas stream a comprising n-butenes;

B) introduction of the feed gas stream a comprising n-butenes and an oxygen-comprising gas into a dehydrogenation reactor and oxidative dehydrogenation of n-butenes to butadiene;

C) cooling and compression of the product gas stream b in at least one cooling stage comprising a quenching column and in a compression stage comprising a compressor, with the product gas stream b being brought into contact with at least one circulated coolant;

D) separation of incondensable and low-boiling gas constituents as gas stream d2 from the gas stream c2 by absorption of the C₄-hydrocarbons in at least one circulated absorption medium;

E) separation of the C₄ product stream d1 by extractive distillation using a solvent which is selective for butadiene into a stream e1 comprising butadiene and the selective solvent and a stream e2 comprising n-butenes;

F) distillation of the stream e1 comprising butadiene and the selective solvent to give a stream f1 consisting essentially of the selective solvent and a stream f2 comprising butadiene;

wherein the measures (i) to (iii) below are carried out
(i) avoidance of formation of explosive gas mixtures by monitoring of the oxygen concentration in the oxygen-comprising gas streams fed into the dehydrogenation zone and control of the mass flows of oxygen-comprising gas streams and gas streams comprising hydrocarbons in such a way that no explosive gas mixtures can be formed;
(ii) interruption of the introduction of the oxygen-comprising gas mixture into the dehydrogenation zone when a limit value for the oxygen concentration in the dehydrogenation gas mixture is exceeded;
(iii) carrying out of the steps A) to F) in apparatuses which are configured so as to be explosion-protected, where liquid-conveying pipes are configured so as to be explosion-protected and gas conduits are configured so as to be detonation-protected.

The invention relates to a process for preparing butadiene by oxidative dehydrogenation of n-butenes.

Butadiene is an important basic chemical and is used, for example, for preparing synthetic rubbers (butadiene homopolymers, styrene-butadiene rubber or nitrile rubber) or for preparing thermoplastic terpolymers (acrylonitrile-butadiene-styrene copolymers). Butadiene is also converted into sulfolane, chloroprene and 1,4-hexamethylenediamine (via 1,4-dichlorobutene and adiponitrile). Vinylcyclohexene which can be dehydrogenated to styrene can also be produced by dimerization of butadiene.

Butadiene can be prepared by thermal dissociation (steam cracking) of saturated hydrocarbons, with naphtha usually being used as raw material. Steam cracking of naphtha gives a hydrocarbon mixture of methane, ethane, ethene, acetylene, propane, propene, propyne, allene, butanes, butenes, butadiene, butynes, methylallene, C₅-hydrocarbons and higher hydrocarbons.

Butadiene can also be obtained by oxidative dehydrogenation of n-butenes (1-butene and/or 2-butene). Any desired mixture comprising n-butenes can be utilized as starting gas mixture for the oxidative dehydrogenation (oxydehydrogenation, ODH) of n-butenes to butadiene. For example, it is possible to use a fraction which comprises n-butenes (1-butene and/or 2-butene) as main constituent and has been obtained from the C₄ fraction from a naphtha cracker by removal of butadiene and isobutene. Furthermore, it is also possible to use gas mixtures which comprise 1-butene, cis-2-butene, trans-2-butene or mixtures thereof and have been obtained by dimerization of ethylene as starting gas. Gas mixtures which comprise n-butenes and have been obtained by fluid catalytic cracking (FCC) can also be used as starting gas.

Process for the oxidative dehydrogenation of butenes to butadiene are basically known.

US 2012/0130137A1, for example, describes a process of this type using catalysts which comprise oxides of molybdenum, bismuth and generally further metals. To maintain the lasting activity of such catalysts for the oxidative dehydrogenation, a critical minimum oxygen partial pressure in the gas atmosphere is necessary in order to avoid excessive reduction and thus a decrease in performance of the catalysts. For this reason, the process can generally also not be carried out using a stoichiometric amount of oxygen or complete conversion of oxygen in the oxydehydrogenation reactor. US 2012/0130137 describes, for example, an oxygen content of 2.5 to 8% by volume in the starting gas.

The necessity of an excess of oxygen for such catalyst systems is generally known and is reflected in the test or process conditions for such catalysts. As representatives, mention may be made of the relatively recent work by Jung et al, (Catal. Surv. Asia 2009, 13, 78-93; DOI 10.1007/s10563-009-9069-5 and Applied Catalysis A; General 2007, 317, 244-249; DOI 10.1016/j.apcata.2006.10.021).

In the case of long pipes (i.e. in the case of which the pipe length is at least about 50 times as great as the pipe diameter), the explosion of a hydrocarbon/oxygen mixture can, at suitable stoichiometric ratios (air ratio lambda=1, ideal mixing), turn into a detonation in a pipe. The change from an explosion into a detonation is, according to the prior art, prevented in the petrochemical industry in combination with a simultaneously explosion-protected construction in pressurized systems by, for example, the following measures:
provision of internals
introduction of beds
installation of explosion protection filters
configuration of the pipes as a bundle
installation of extinguishing systems (powder)
liquid mist

Such internals bring the explosion due to a high mass to a stop at small gap widths and a large surface area. The spreading flame is cooled and is finally extinguished in the ideal case.

Small gap dimensions at a large surface area are common to all these measures. However, the hydrocarbon mixture flowing through in processes for the oxidative dehydrogenation of butenes to butadiene intentionally comprises not inconsiderable concentrations of 1,3-butadiene. This material has the property of polymerizing in gaps and on surfaces. The use of the abovementioned protective measures thus does not lead to the objective in a process for preparing 1,3-butadiene since the operating time until complete covering/blockage of the internals by deposits, caused by polymerization, is too short.

It is an object of the invention to provide a process for preparing butadiene from n-butenes which is very safe. An appropriate safety concept has to ensure that persons and the surroundings are not exposed to a hazard.

The object is achieved by a process for preparing butadiene from n-butenes, which comprises the following steps:

A) provision of a feed gas stream a comprising n-butenes;

B) introduction of the feed gas stream a comprising n-butenes and an oxygen-comprising gas into at least one dehydrogenation zone comprising a dehydrogenation reactor and oxidative dehydrogenation of n-butenes to butadiene, giving a product gas stream b comprising butadiene, unreacted n-butenes, water vapor, oxygen, low-boiling hydrocarbons, possibly carbon oxides and possibly inert gases;

C) cooling and compression of the product gas stream b in at least one cooling stage comprising at least one quenching column and in at least one compression stage comprising at least one compressor and optionally one or more intermediate cooling units between the compressors, with the product gas stream b being brought into contact with at least one circulated coolant to give at least one condensate stream c1 comprising water and a gas stream c2 comprising butadiene, n-butenes, water vapor, oxygen, low-boiling hydrocarbons, possibly carbon oxides and possibly inert gases;

D) separation of incondensable and low-boiling gas constituents comprising oxygen, low-boiling hydrocarbons, possibly carbon oxides and possibly inert gases as gas stream d2 from the gas stream c2 by absorption of the C₄-hydrocarbons comprising butadiene and n-butenes in at least one circulated absorption medium, giving at least one absorption medium stream loaded with C₄-hydrocarbons and the gas stream d2, and subsequent desorption of the C₄-hydrocarbons from the loaded absorption medium stream to give a C₄ product gas stream d1;

E) separation of the C₄ product stream d1 by extractive distillation using a solvent which is selective for butadiene into a stream e1 comprising butadiene and the selective solvent and a stream e2 comprising n-butenes;

F) distillation of the stream e1 comprising butadiene and the selective solvent to give a stream f1 consisting essentially of the selective solvent and a stream f2 comprising butadiene;

wherein the measures (i) to (iii) below are carried out:

(i) avoidance of formation of explosive gas mixtures by monitoring of the oxygen concentration in the oxygen-comprising gas streams fed into the dehydrogenation zone and control of the mass flows of oxygen-comprising gas streams and gas streams comprising hydrocarbons in such a way that no explosive gas mixtures can be formed;

(ii) interruption of the introduction of the oxygen-comprising gas mixture into the dehydrogenation zone when a limit value for the oxygen concentration in the dehydrogenation gas mixture is exceeded;

(iii) carrying out of the steps A) to F) in apparatuses which are configured so as to be explosion-protected, where liquid-conveying pipes are configured so as to be explosion-protected and gas conduits are configured so as to be detonation-protected.

According to the invention, the safety of the process is ensured by a bundle of measures. Firstly, a safety-oriented shutdown of the plant to maintain the internal integrity of the plant (internals) is provided. Thus, the sometimes fluctuating oxygen concentration in the oxygen-comprising gas streams which are fed into the dehydrogenation reactor is monitored by means of suitable on-line analysis. Furthermore, the mass flows of oxygen-comprising gas streams and hydrocarbon-comprising gas streams are monitored by means of certified quantity measurements with appropriate ratio regulation. This ensures maintenance of a minimum distance between the prevailing oxygen concentration in the process streams and the lower explosive limit, taking account of operational fluctuations and the accuracy of the on-line analysis. When the limit values of the oxygen concentration at the lower explosive limit are exceeded, the reactor or the entire plant is shut down.

For the purposes of the present invention, “shutdown of the reactor” is interruption of the feed gas streams to the reactor, but at least interruption of the supply of oxygen to the reactor, e.g. by closure of valves in the pipes concerned.

For the purposes of the present invention, “shut down of the entire plant” is interruption of all relevant feed gas streams into the plant, of product streams from the plant and of streams within the plant, e.g. by closure of valves in the pipes concerned and optionally by carrying out further measures to bring the plant into a safe state.

Furthermore, gas-conveying apparatuses are configured so as to be explosion-protected in the structural assemblies affected by a risk of explosion and gas-conveying pipes are configured so as to be detonation-protected in order to ensure the integrity of the plant relative to the surroundings. Thus, gas-conveying apparatuses and liquid-conveying pipes are configured so as to be explosion-protected in the structural assemblies concerned. In contrast to the prior art, gas-conveying pipes which have an unobstructed length which is at least 50 times their diameter are designed so as to be detonation-protected. The combination of the explosion-protected design of apparatuses, through which gas flows and which are “short” in the sense of a short flow length, and the detonation-protected design of “long” pipes leads to an inherently safe design of the plant. Lengths and diameters of the apparatuses through which gas flows are selected so that detonation can be ruled out. In addition, internals (trays) in distillation columns are configured so as to be gastight. Gas feed lines are, if possible in process engineering terms, made so as to dip into the liquid in order to prevent propagation of an explosion in the pipe into the attached plant components. Coolers are preferably configured as direct coolers instead of heat exchangers.

Preference is given to designing one or more of the following apparatuses so as to be explosion-protected;
the dehydrogenation reactor used in step B);
the quenching column used in step C);
the compressors used in step C);
the intermediate coolers used in step C);
the distillation column used in step F);
the decanter used in the solvent regeneration unit.

As regards an explosion-protected configuration, the following may be said: an explosion is uncontrolled burning of an ignitable mixture with a laminar flame front. In the case of stoichiometric air/hydrocarbon mixtures, a maximum explosion pressure rise by a factor of 10 is calculated.

Thus, an explosion-protected configuration means that the apparatus or the pipe is designed for a pressure rise by up to a factor of 10.

As regards a detonation-protected configuration, the following may be said: an explosion can after a flow length and at a minimum concentration of fuel and oxygen turn into a detonation. This happens when the flame front accelerates at more than the speed of sound. The pressure rise factor can be 50 and more.

Detonation can be ruled out by suitable selection of the ratio of length:diameter of a gas-conveying pipe and an apparatus. This ratio is generally allowed to be not more than 50:1. If the ratio is more than 50:1, the pipe has a detonation-protected configuration.

Thus, according to the invention, gas conduits between the apparatuses of steps A) to F) either have such a ratio of length to diameter that detonation is ruled out or the gas-conveying pipes concerned are designed so as to be detonation-protected. This ratio is generally not more than 50:1. At a higher ratio, the conduits are designed so as to be detonation-protected. Pipes affected are, for example, designed for 50 times the maximum operating pressure. At an operating pressure of 5 bar, a design pressure of, for example, 250 bar is calculated. The material and the wall thickness of the pipe are then to be selected in accordance with this pressure (=mechanical design).

The intermediate coolers used in step C) are preferably configured as direct coolers. These direct coolers are configured as vertical columns having internals. Possible internals are beds such as Pall rings, packings or preferably column trays such as sieve trays, cascade trays or valve trays. A liquid cooling medium such as water or a hydrocarbon such as toluene, preferably mesitylene, is conveyed in cocurrent with or preferably in countercurrent to the gas stream to be cooled.

The distillation columns used in steps D), E) and/or F) are preferably configured as tray columns having gastight column trays.

Furthermore, gas feed lines in the distillation columns used in steps D), E) and/or F) preferably dip into the liquid.

Gas conduits between the apparatuses of steps A) to F) have such a ratio of length:diameter that detonation is impossible. The ratio of length:diameter is preferably not more than 50:1.

As coolant in step C), it is possible to use water, alkaline aqueous solutions, organic solvents or mixtures thereof. Preference is given to using an organic solvent. Aromatic hydrocarbon solvents such as toluene, xylene or mesitylene are preferred.

The embodiments below are preferred or particularly preferred variants of the process of the invention:

Step C) comprises at least one cooling stage Ca) and one compression stage Cb). Stage Ca) is preferably carried out in a plurality of stages Ca1) to Can), in particular in two stages Ca1) and Ca2). In one variant, at least part of the coolant which has passed through the second stage Ca2) is fed as coolant to the first stage Ca1).

Stage Cb) generally comprises at least one compression stage Cba) and at least one cooling stage Cbb). The gas compressed in the compression stage Cba) is preferably brought into contact with a coolant in the at least one cooling stage Cbb). The coolant for the cooling stage Cbb) particularly preferably comprises the same organic solvent used as coolant in stage Ca). In a particularly preferred variant, at least part of this coolant which has passed through the at least one cooling stage Cbb) is fed as coolant to the stage Ca).

The stage Cb) preferably comprises a plurality of compression stages Cba1) to Cban) and cooling stages Cbb1) to Cbbn), for example four compression stages Cba1) to Cba4) and four cooling stages Cbb1) to Cbb4).

The compression stages Cba1) to Cban) are preferably all configured so as to be explosion-protected. The cooling stages Cbb1) to Cbbn) are preferably all designed as direct cooling units.

Step D) preferably comprises the steps Da) to Dc):

Da) absorption of the C₄-hydrocarbons comprising butadiene and n-butenes in a high-boiling absorption medium to give an absorption medium stream loaded with C₄-hydrocarbons and the gas stream d2,

Db) removal of oxygen from the absorption medium stream loaded with C₄-hydrocarbons from step Da) by stripping with an incondensable gas stream and

Dc) desorption of the C₄-hydrocarbons from the loaded absorption medium stream to give a C₄ product gas stream d1. This then preferably has an oxygen content of less than 100 ppm.

In one embodiment, the absorption medium used in step D) is the same organic solvent as a coolant used in step C), with at least part of this absorption medium being, after desorption of the C₄-hydrocarbons, fed as coolant to step C). In a preferred variant of this embodiment, the absorption medium and coolant is toluene, xylene or mesitylene.

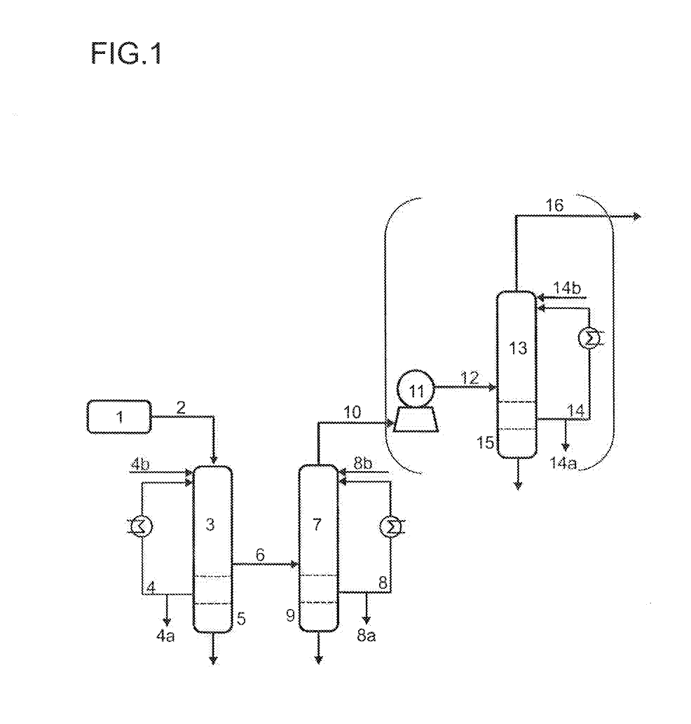

Preferred embodiments of the process are shown in FIGS. 1 to 3 and are described in detail below.

In a step A), a feed gas stream comprising n-butenes is provided.

As feed gas stream, it is possible to use pure n-butenes (1-butene and/or cis-/trans-2-butene) or gas mixtures comprising butenes. A gas mixture of this type can be obtained, for example, by nonoxidative dehydrogenation of n-butane. It is also possible to use a fraction which comprises n-butenes (1-butene and cis-/trans-2-butene) as main constituent and has been obtained from the C₄ fraction from naphtha cracking by removal of butadiene and isobutene. Furthermore, it is also possible to use gas mixtures which comprise pure 1-butene, cis-2-butene, trans-2-butene or mixtures thereof and have been obtained by dimerization of ethylene as starting gas. Gas mixtures which comprise n-butenes and have been obtained by fluid catalytic cracking (FCC) can also be used as starting gas.

In an embodiment of the process of the invention, the starting gas mixture comprising n-butenes is obtained by nonoxidative dehydrogenation of n-butane. A high yield of butadiene, based on n-butane used, can be obtained by the coupling of a nonoxidative catalytic dehydrogenation with the oxidative dehydrogenation of the n-butenes formed. The nonoxidative catalytic dehydrogenation of n-butane gives a gas mixture comprising butadiene, 1-butene, 2-butene and unreacted n-butane and also secondary constituents. Usual secondary constituents are hydrogen, water vapor, nitrogen, CO and CO₂, methane, ethane, ethene, propane and propene. The composition of the gas mixture leaving the first dehydrogenation zone can vary greatly depending on the way in which the dehydrogenation is carried out. Thus, when the dehydrogenation is carried out with introduction of oxygen and additional hydrogen, the product gas mixture has a comparatively high content of water vapor and carbon oxides. In modes of operation without introduction of oxygen, the product gas mixture from the nonoxidative dehydrogenation has a comparatively high content of hydrogen.

In step B), the feed gas stream comprising n-butenes and an oxygen-comprising gas are introduced into at least one dehydrogenation zone 1 (ODH reactor) and the butenes comprised in the gas mixture are oxidatively dehydrogenated to butadiene in the presence of an oxydehydrogenation catalyst.

Catalysts suitable for the oxydehydrogenation are generally based on an Mo—Bi—O-comprising multimetal oxide system which generally additionally comprises iron. In general, the catalyst system comprises further additional components, for example potassium, cesium, magnesium, zirconium, chromium, nickel, cobalt, cadmium, tin, lead, germanium, lanthanum, manganese, tungsten, phosphorus, cerium, aluminum or silicon. Iron-comprising ferrites have also been proposed as catalysts.

In a preferred embodiment, the multimetal oxide comprises cobalt and/or nickel. In a further preferred embodiment, the multimetal oxide comprises chromium. In a further preferred embodiment, the multimetal oxide comprises manganese.

Examples of Mo—Bi—Fe—O-comprising multimetal oxides are Mo—Bi−Fe—Cr-O- or Mo—Bi—Fe—Zr—O-comprising multimetal oxides. Preferred systems are, for example, described in U.S. Pat. No. 4,547,615 (Mo₁₂BiFe_{0.1}Ni₈ZrCr₃K_{0.2}Oₓ and Mo₁₂BiFe_{0.1}Ni₈AlCr₃K_{0.2}Oₓ), U.S. Pat. No. 4,424,141 (Mo₁₂BiFe₃Co_{4.5}Ni_{2.5}P_{0.5}K_{0.1}Oₓ+SiO₂), DE-A 25 30 959 (Mo₁₂BiFe₃Co_{4.5}N_{12.5}Cr_{0.5}K_{0.1}Oₓ, Mo_{13.75}BiFe₃Co_{4.5}Ni_{2.5}Ge_{0.5}K_{0.8}Oₓ, Mo₁₂BiFe₃Co_{4.5}Ni_{2.5}Mn_{0.5}K_{0.1}Oₓ and Mo₁₂BiFe₃Co_{4.5}Ni_{2.5}La_{0.5}K_{0.1}Oₓ), U.S. Pat. No. 3,911,039 (Mo₁₂BiFe₃Co_{4.5}Ni_{2.5}Sn_{0.5}K_{0.1}Oₓ), DE-A 25 30 959 and DE-A 24 47 825 (Mo₁₂BiFe₃Co_{4.5}N_{2.5}W_{0.5}K_{0.1}Oₓ).

Suitable multimetal oxides and their preparation are also described in U.S. Pat. No. 4,423,281 (Mo₁₂BiNi₈Pb_{0.5}Cr₃K_{0.2}Oₓ and Mo₁₂Bi_{b}Ni₇Al₃Cr_{0.5}K_{0.5}Oₓ), U.S. Pat. No. 4,336,409 (Mo₁₂BiNi₆Cd₂Cr₃P_{0.5}Oₓ), DE-A 26 00 128 (Mo₁₂BiNi_{0.5}Cr₃P_{0.5}Mg_{7.5}K_{0.1}Oₓ+SiO₂) and DE-A 24 40 329 (Mo₁₂BiCo_{4.5}Ni_{2.5}Cr₃P_{0.5}K_{0.1}Oₓ).

Particularly preferred catalytically active multimetal oxides comprising molybdenum and at least one further metal have the general formula (Ia):

Mo₁₂BiₐFe_{b}Co_{c}Ni_{d}CrₑX¹_{f}X²_{g}O_{y} (Ia),

where

X¹=Si, Mn and/or Al,

X²=Li, Na, K, Cs and/or Rb,

0.2≤a≤1,

0.5≤b≤10,

0≤c≤10,

0≤d≤10,

2≤c+d≤10,

0≤e≤2,

0≤f≤10,

0≤g≤0.5,

y=a number determined by the valence and abundance of elements other than oxygen in (Ia) so as to maintain charge neutrality.

Preference is given to catalysts whose catalytically active oxide composition has only Co from among the two metals Co and Ni (d=0). X¹ is preferably Si and/or Mn and X² is preferably K, Na and/or Cs, with particular preference being given to X²=K.

The gas comprising molecular oxygen generally comprises more than 10% by volume, preferably more than 15% by volume and even more preferably more than 20% by volume, of molecular oxygen. It is preferably air. The upper limit to the content of molecular oxygen is generally 50% by volume or less, preferably 30% by volume or less and even more preferably 25% by volume or less. In addition, any inert gases can be comprised in the gas comprising molecular oxygen. As possible inert gases, mention may be made of nitrogen, argon, neon, helium, CO, CO₂ and water. The amount of inert gases is generally 90% by volume or less, preferably 85% by volume or less and even more preferably 80% by volume or less, in the case of nitrogen. In the case of constituents other than nitrogen, it is generally 10% by volume or less, preferably 1% by volume or less,

To carry out the oxidative dehydrogenation with complete conversion of n-butenes, preference is given to a gas mixture which has a molar oxygen:n-butenes ratio of at least 0.5. Preference is given to working at an oxygen:n-butenes ratio of from 0.55 to 10. To set this value, the starting gas can be mixed with oxygen or an oxygen-comprising gas, for example air, and optionally additional inert gas or steam. The oxygen-comprising gas mixture obtained is then fed to the oxydehydrogenation.

The reaction temperature of the oxydehydrogenation is generally controlled by means of a heat transfer medium which is present around the reaction tubes. Possible liquid heat transfer media of this type are, for example, melts of salts such as potassium nitrate, potassium nitrite, sodium nitrite and/or sodium nitrate and also melts of metals such as sodium, mercury and alloys of various metals. However, ionic liquids or heat transfer oils can also be used. The temperature of the heat transfer medium is in the range from 220 to 490° C. and preferably in the range from 300 to 450° C. and particularly preferably in the range from 350 to 420° C.

Owing to the exothermic nature of the reactions which occur, the temperature in particular sections of the interior of the reactor can be higher than that of the heat transfer medium during the reaction and a hot spot is formed. The position and magnitude of the hot spot is determined by the reaction conditions, but can also be regulated by the dilution ratio of the catalyst zone or the flow of mixed gas through it. The difference between hot spot temperature and the temperature of the heat transfer medium is generally 1-150° C., preferably 10-100° C. and particularly preferably 20-80° C. The temperature at the end of the catalyst bed is generally 0-100° C. above, preferably 0.1-50° C. above, particularly preferably 1-25° C. above, the temperature of the heat transfer medium.

The oxydehydrogenation can be carried out in all fixed-bed reactors known from the prior art, for example in a tray oven, a fixed-bed tube reactor or shell-and-tube reactor or in a plate heat exchanger reactor. A shell-and-tube reactor is preferred.

The reactor is designed so as to be explosion-protected. Here, the wall of the reactor is designed in accordance with the explosion pressure to be expected. For example, the maximum operating pressure of the reactor is 5 bar and the explosion pressure factor of the gas mixture present in the reactor is 10. This gives a design pressure of 50 bar for the mechanical design of the reactor wall. Both material and wall thickness are then selected according to the design pressure of 50 bar.

The oxidative dehydrogenation is preferably carried out in fixed-bed tube reactors or fixed-bed shell-and-tube reactors. The reaction tubes are (like the other elements of the shell-and-tube reactor) generally made of steel. The wall thickness of the reaction tubes is typically from 1 to 3 mm. Their internal diameter is generally (uniformly) from 10 to 50 mm or from 15 to 40 mm, frequently from 20 to 30 mm. The number of reaction tubes accommodated in the shell-and-tube reactor is generally at least 1000, or 3000, or 5000, preferably at least 10000. The number of reaction tubes accommodated in the shell-and-tube reactor is frequently from 15000 to 30000 or up to 40000 or up to 50000. The length of the reaction tubes is normally a few meters, with a reaction tube length in the range from 1 to 8 m, frequently from 2 to 7 m, often from 2.5 to 6 m, being typical.

Furthermore, the catalyst bed installed in the reactor 1 can consist of a single zone or of 2 or more zones. These zones can consist of pure catalyst or be diluted with a material which does not react with the starting gas or components of the product gas of the reaction. Furthermore, the catalyst zones can consist of all-active catalysts or supported coated catalysts.

The product gas stream 2 leaving the oxidative dehydrogenation generally comprises not only butadiene but also unreacted 1-butene and 2-butene, oxygen and water vapor. It also generally comprises carbon monoxide, carbon dioxide, inert gases (mainly nitrogen), low-boiling hydrocarbons such as methane, ethane, ethene, propane and propene, butane and isobutane, possibly hydrogen and possibly oxygen-comprising hydrocarbons, known as oxygenates, as secondary components. Oxygenates can be, for example, formaldehyde, furan, acetic acid, maleic anhydride, formic acid, methacrolein, methacrylic acid, crotonaldehyde, crotonic acid, propionic acid, acrylic acid, methyl vinyl ketone, styrene, benzaldehyde, benzoic acid, phthalic anhydride, fluorenone, anthraquinone and butyraldehyde.

The product gas stream 2 at the reactor outlet has a temperature close to the temperature at the end of the catalyst bed. The product gas stream is then brought to a temperature of 150 to 400° C., preferably 160 to 300° C., particularly preferably 170 to 250° C. It is possible to insulate the line through which the product gas stream flows in order to keep the temperature in the desired range, but preference is given to using a heat exchanger. This heat exchanger system can be of any type as long as this system enables the temperature of the product gas to be kept at the desired level. Examples of suitable heat exchangers are coil heat exchangers, plate heat exchanger, double-tube heat exchangers, multitube heat exchangers, boiler coil heat exchangers, boiler wall heat exchangers, liquid-liquid contact heat exchangers, air heat exchangers, direct contact heat exchangers and finned tube heat exchangers. Since part of the high-boiling by-products present in the product gas can condense out while the temperature of the product gas is set to the desired temperature, the heat exchanger system should preferably have two or more heat exchangers. If two or more heat exchangers provided are arranged in parallel and distributed cooling of the product gas in the heat exchangers is made possible, the amount of high-boiling by-products which deposit in the heat exchangers decreases and their operating time can thus be increased. As an alternative to the abovementioned method, the two or more heat exchangers provided can be arranged in parallel. The product gas is fed to one or more but not all of the heat exchangers which are relieved by other heat exchangers after a particular operating time. In the case of this method, cooling can be continued, part of the heat of reaction can be recovered and, in parallel thereto, the high-boiling by-products deposited in one of the heat exchangers can be removed. As an organic solvent of the type mentioned above, it is possible to use any solvent without restriction as long as it is able to dissolve the high-boiling by-products; for example, an aromatic hydrocarbon solvent such as toluene, xylene, etc., or an alkaline aqueous solvent such as the aqueous solution of sodium hydroxide can be used for this purpose.

Subsequently, in step C), a major part of the high-boiling secondary components and of the water can be separated off from the product gas stream 2 by cooling. This cooling and separation is preferably effected in a quench. This quench can consist of one stage (3 in FIG. 1) or a plurality of stages (3, 7 in FIG. 1). Preference is given to using a process in which the product gas stream 2 is brought into contact directly with the coolant 4 and is cooled thereby. As coolant, it is possible to use water, alkaline aqueous solutions, organic solvents or a combination or mixture thereof. Preference is given to using an organic solvent. As solvent it is possible to use any solvent as long as it is capable of taking up parts of the secondary components present in the gas stream. Particular preference is given to solvents such as toluene, xylene or mesitylene.

In a preferred embodiment of the process of the invention, toluene is used as coolant. In a particularly preferred embodiment of the process of the invention, mesitylene is used as coolant.

Preference is given to a two-stage quench. The cooling temperature of the product gas differs depending on the temperature of the product gas 2 obtained from the reactor outlet and of the coolant 4. In general, the product gas 2 has, depending on the presence and temperature level of a heat exchanger upstream of the quench inlet, a temperature of 100-440° C. The product gas inlet into the quench has to be designed so that blockage by deposits at and directly before the gas inlet is minimized or prevented. The product gas is brought into contact with the coolant in the 1^{st} quenching stage 3. Here, the coolant can be introduced through a nozzle in order to achieve very efficient mixing with the product gas. For the same purpose, internals such as further nozzles through which the product gas and the coolant have to pass together can be installed in the quenching stage. The coolant inlet into the quench has to be designed so that blockage by deposits in the region of the coolant inlet is minimized or prevented.

In general, the product gas 2 is cooled to 5-180° C., preferably to 30-130° C. and even more preferably to 60-110° C., in the first quenching stage. The temperature of the cooling medium 4 at the inlet can generally be 25-200° C., preferably 40-120° C., in particular 50-90° C. The pressure in the first quenching stage is not subject to any particular restrictions but is generally 0.01-4 bar (gauge), preferably 0.1-2 bar (gauge) and particularly preferably 0.2-1 bar (gauge). When a large amount of high-boiling by-products is present in the product gas, polymerization of the high-boiling by-products and deposition of solids caused by high-boiling by-products in this process section can easily occur. The coolant 4 used in the cooling tower of the first quenching stage is circulated. The circulating flow of the coolant in liters per hour based on the mass flow of butadiene in grams per hour can generally be 0.0001-5 l/g, preferably 0.001-1 l/g and particularly preferably 0.002-0.2 l/g.

The temperature of the coolant 4 at the bottom can generally be 27-210° C., preferably 45-130° C., in particular 55-95° C. Since the loading of the coolant 4 with secondary components increases over the course of time, part of the loaded coolant (4a) can be taken off from the circuit and the circulating amount can be kept constant by addition of unloaded coolant (4b). The ratio of amount discharged and amount added depends on the vapor loading of the product gas and the product gas temperature at the end of the first quenching stage. Depending on temperature, pressure and water content of the product gas 2, condensation of water can occur in the first quenching stage 3. In this case, an additional aqueous phase 5 which can additionally comprise water-soluble secondary components can be formed. This can then be taken off at the bottom of the quenching stage 3. Preference is given to a mode of operation in which no aqueous phase is formed in the first quenching stage 3.

The cooled product gas stream 6, which may have been depleted in secondary components, can then be fed to a second quenching stage 7. In this, it can again be brought into contact with a coolant 8.

As coolant 8, it is possible to use water, alkaline aqueous solutions, organic solvents or a combination or mixtures thereof. Preference is given to using an organic solvent. As a solvent of the type mentioned above, it is possible to use any solvent as long as it is capable of taking up parts of the secondary components present in the gas stream. Preference is given to aromatic hydrocarbon solvents such as toluene or mesitylene since the solubility limit in these is greater than 1000 ppm, i.e. mg of active oxygen/kg of solvent.

In general, the product gas is cooled to from 5 to 100° C., preferably to 15-85° C. and even more preferably to 30-70° C., up to the gas outlet from the second quenching stage 7. The coolant can be introduced in countercurrent to the product gas. In this case, the temperature of the cooling medium 8 at the coolant inlet can be 5-100° C., preferably 15-85° C., in particular 30-70° C. The pressure in the second quenching stage 7 is not subject to any particular restrictions but is generally 0.01-4 bar (gauge), preferably 0.1-2 bar (gauge) and particularly preferably 0.2-1 bar (gauge). The coolant 8 used in the cooling tower of the second quenching stage is circulated. The circulating flow of the coolant 8 in liters per hour based on the mass flow of butadiene in grams per hour can generally be 0.0001-5 l/g, preferably 0.001-1 l/g and particularly preferably 0.002-0.2 l/g.

Depending on the temperature, pressure and water content of the product gas 6, condensation of water can occur in the second quenching stage 7. In this case, an additional aqueous phase 9, which can additionally comprise water-soluble secondary components, can be formed. This can then be taken off at the bottom of the quenching stage 7. If an aqueous phase 9 is present at the bottom of the second quenching stage 7 or water is used as coolant in part of the quench, the dissolution of by-products of the ODH reaction, for example acetic acid, maleic anhydride, etc., occurs better at an elevated pH than at a low pH. Since the dissolution of by-products such as those mentioned above lowers the pH of, for example, water, the pH can be kept constant or increased by addition of an alkaline medium. In general, the pH of the aqueous phase at the bottom of the second quenching stage 7 is maintained at 1-14, preferably 2-12, particularly preferably 3-11. The more acidic the value, the less alkaline medium has to be introduced. The more basic, the better the dissolution of some by-products. However, very high pH values lead to dissolution of by-products such as CO₂ and thus to a very high consumption of the alkaline medium. The temperature of the coolant 8 at the bottom can generally be 20-210° C., preferably 35-120° C., in particular 45-85° C.

Since the loading of the coolant 8 with secondary components increases over the course of time, part of the loaded coolant (8a) can be taken off from the circuit and the circulating amount can be kept constant by addition of unloaded coolant (8b).

To achieve very good contact of product gas and coolant, internals can be present in the second quenching stage. Such internals comprise, for example, bubble cap trays, centrifugal trays and/or sieve trays, columns having structured packings, e.g. sheet metal packings having a specific surface area of from 100 to 1000 m²/m³, e.g. Mellapak® 250 Y, and columns packed with random packing elements.

The circuits of the two quenching stages can be either separate from one another or connected to one another. Thus, for example, stream 8a can be added to the stream 4b or replace the latter. The desired temperature of the circulating streams can be set via suitable heat exchangers.

To minimize entrainment of liquid constituents from the quench into the offgas line, it is possible to undertake suitable constructional measures, for example the installation of a demister. Furthermore, high-boiling substances which are not separated off from the product gas in the quench can be removed from the product gas by means of further constructional measures, for example further gas scrubs. A gas stream 10 in which n-butane, 1-butene, 2-butenes, butadiene, possibly oxygen, hydrogen, water vapor, small amounts of methane, ethane, ethene, propane and propene, isobutane, carbon oxides, inert gases and parts of the solvent used in the quench remain is obtained. Furthermore, traces of high-boiling components which have not been separated off quantitatively in the quench can remain in this product gas stream.

The product gas stream 10 from the quench is subsequently compressed in at least one compression stage 11 and then cooled further in the cooling stage 13, with at least one condensate stream comprising water 15 and the solvent 14 used in the quench condensing out and a gas stream 16 comprising butadiene, 1-butene, 2-butenes, oxygen, water vapor, possibly low-boiling hydrocarbons such as methane, ethane, ethene, propane and propene, butane and isobutane, possibly carbon oxides and possibly inert gases remaining. Furthermore, traces of high-boiling components can remain in this product gas stream.

The compression and cooling of the gas stream 10 can be carried out in one or more stages (n stages). In general, the gas stream is compressed overall from a pressure in the range from 1.0 to 4.0 bar (absolute) to a pressure in the range from 3.5 to 20 bar (absolute). Each compression stage is followed by a cooling stage in which the gas stream is cooled to a temperature in the range from 15 to 60° C. The condensate stream can thus comprise a plurality of streams in the case of multistage compression, The condensate stream comprises largely water 15 and the solvent 16 used in the quench. Both streams can additionally comprise small amounts of low boilers, C₄-hydrocarbons, oxygenates and carbon oxides.

The cooling stages are preferably configured as direct coolers. These direct coolers are configured as vertical columns having internals. Possible internals are beds such as Pall rings, packings or preferably column trays such as sieve trays, cascade trays or valve trays. A liquid cooling medium such as water or a hydrocarbon such as toluene, preferably mesitylene, is conveyed in cocurrent with or preferably in countercurrent to the gas stream to be cooled.

To cool the stream 12 and/or to remove further secondary components from the stream 12, the condensed solvent 14 used in the quench can be cooled in a heat exchanger and recirculated to the cooling stage 13. Since the loading of the coolant 14 with secondary components increases over the course of time, part of the loaded coolant 14a can be taken off from the circuit and the circulated amount can be kept constant by addition of unloaded coolant 14b.

The condensate stream 14a can be recirculated to the circulating stream 4b and/or 8b of the quench. In this way, the C₄ components absorbed in the condensate stream 14a can be reintroduced into the gas stream and the yield can thus be increased.

Suitable compressors are, for example, turbocompressors, rotary piston compressors and reciprocating piston compressors. The compressors can be driven by, for example, an electric motor, an expander or a gas or steam turbine. Typical compression ratios (exit pressure:entry pressure) per compressor stage are, depending on the construction type, in the range from 1.5 to 3.0. The cooling of the compressed gas is effected by means of heat exchangers which can be configured, for example, as shell-and-tube, coil or plate heat exchangers or direct cooling units.

The housings of the compressors preferably have an explosion-protected design. Here, the housings of the compressors are designed in accordance with the explosion pressures to be expected. For example, the maximum operating pressure of a compressor is 5 bar and the explosion pressure factor of the gas mixture present in the compressor is 10. This gives a design pressure of 50 bar for the mechanical design of the compressor housing. Both material and wall thickness are then selected according to the design pressure of 50 bar.

The gas stream 16 comprising butadiene, n-butenes, oxygen, low-boiling hydrocarbons (methane, ethane, ethene, propane, propene, n-butane, isobutane), possibly water vapor, possibly carbon oxides and possibly inert gases is fed as starting stream to the further work-up.

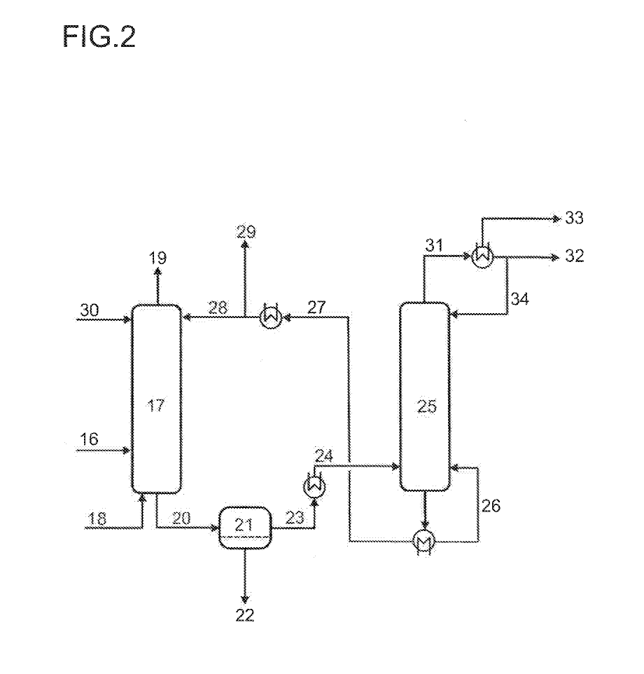

In a step D) (FIG. 2), incondensable and low-boiling gas constituents comprising oxygen, low-boiling hydrocarbons (methane, ethane, ethene, propane, propene), carbon oxides and inert gases are separated off as gas stream 19 from the process gas stream 16 by absorption of the C₄-hydrocarbons in a high-boiling absorption medium (28 and/or 30) in an absorption column 17 and subsequent desorption of the C₄-hydrocarbons. This step D) preferably comprises the substeps

Da) absorption of the C₄-hydrocarbons comprising butadiene and n-butenes in a high-boiling absorption medium (28 and/or 30) to give an absorption medium stream loaded with C₄-hydrocarbons and the gas stream 19,

Db) removal of oxygen from the absorption medium stream loaded with C₄-hydrocarbons from step Da) by stripping with an incondensable gas stream 18 to give an absorption medium stream 20 loaded with C₄-hydrocarbons and

Dc) desorption of the C₄-hydrocarbons from the loaded absorption medium stream to give a C₄-product gas stream 31.

For this purpose, the gas stream 16 is brought into contact with an inert absorption medium in the absorption stage 17 and the C₄-hydrocarbons are absorbed in the inert absorption medium, giving an absorption medium 20 loaded with C₄-hydrocarbons and an offgas 19 comprising the remaining gas constituents. In a desorption stage, the C₄-hydrocarbons are liberated again from the high-boiling absorption medium.

The absorption stage can be carried out in any suitable absorption column known to those skilled in the art. The absorption can be effected by simply passing the product gas stream through the absorption medium. However, it can also be carried out in columns or in rotational absorbers. It can be carried out in cocurrent, countercurrent or cross-current. The absorption is preferably carried out in countercurrent. Suitable absorption columns are, for example, tray columns having bubble cap trays, centrifugal trays and/or sieve trays, columns having structured packings, e.g. sheet metal packings having a specific surface area of from 100 to 1000 m²/m³, e.g. Mellapak® 250 Y, and columns packed with random packing elements. However, trickle towers and spray towers, graphite block absorbers, surface absorbers such as thick film and thin film absorbers and also rotational columns, plate scrubbers, crossed spray scrubbers and rotational scrubbers are also possible.

The absorption column is preferably a tray column which is designed so as to be explosion-protected. Here, the wall of the column is designed in accordance with the explosion pressure to be expected. For example, the maximum operating pressure of the column is 10 bar and the explosion pressure factor of the gas mixture present in the column is 9. This gives a design pressure of 90 bar for the mechanical design of the column wall. Both material and wall thickness are then selected according to the design pressure of 90 bar.

The absorption column preferably has gastight column trays. The gas-conveying feed lines into the absorption column preferably dip into the liquid. This prevents propagation of the flame front into the column.

In one embodiment, the gas stream 16 comprising butadiene, n-butenes and the low-boiling and incondensable gas constituents is fed into the lower region of an absorption column. In the upper region of the absorption column, the high-boiling absorption medium (28 and/or 30) is introduced.

Inert absorption media used in the absorption stage are generally high-boiling nonpolar solvents in which the C₄-hydrocarbon mixture to be separated off has a significantly greater solubility than the remaining gas constituents to be separated off. Suitable absorption media are comparatively nonpolar organic solvents, for example aliphatic C₈-C₁₈-alkanes, or aromatic hydrocarbons such as the middle oil fractions from paraffin distillation, toluene or ethers having bulky groups, or mixtures of these solvents, with a polar solvent such as 1,2-dimethyl phthalate being able to be added to these. Further suitable absorption media are esters of benzoic acid and phthalic acid with straight-chain C₁-C₈-alkanols and also heat transfer oils such as biphenyl and diphenyl ether, chloro derivatives thereof and triarylalkenes. One suitable absorption medium is a mixture of biphenyl and diphenyl ether, preferably having the azeotropic composition, for example the commercially available Diphyl®. This solvent mixture frequently comprises dimethyl phthalate in an amount of from 0.1 to 25% by weight.

Preferred absorption media are solvents which have a solvent capability for organic peroxides of at least 1000 ppm (mg of active oxygen/kg of solvent). In a preferred embodiment, toluene, xylene or mesitylene is used as solvent for the absorption.

An offgas stream 19 comprising essentially oxygen, low-boiling hydrocarbons (methane, ethane, ethene, propane, propene), possibly C₄-hydrocarbons (butane, butenes, butadiene), possibly inert gases, possibly carbon oxides and possibly water vapor is taken off at the top of the absorption column 17. This stream can be partly fed to the ODH reactor. This enables, for example, the feed stream to the ODH reactor to be set to the desired C₄-hydrocarbon content.

At the bottom of the absorption column, residues of oxygen dissolved in the absorption medium are discharged by flushing with a gas 18 in a further column. The remaining proportion of oxygen is preferably so low that the stream 31 which leaves the desorption column and comprises butane, butene and butadiene comprises a maximum of 100 ppm of oxygen.

The stripping-out of the oxygen can be carried out in any suitable column known to those skilled in the art. Stripping can be effected by simply passing incondensable gases through the loaded absorption solution. C₄-hydrocarbons which are also stripped out is scrubbed back into the absorption solution in the upper part of the absorption column 17 by the gas stream being fed back into this absorption column. This can be effected both by providing the stripper column with tubes and by direct installation of the stripper column below the absorber column. Since the pressure in the stripping column section and the absorption column section is identical according to the invention, this can be achieved by direct coupling. Suitable stripping columns are, for example, tray columns having bubble cap trays, centrifugal trays and/or sieve trays, columns having structured packings, e.g. sheet metal packings having a specific surface area of from 100 to 1000 m²/m³, e.g. Mellapak® 250 Y, and columns packed with random packing elements. However, trickle towers and spray towers and also rotational columns, plate scrubbers, crossed spray scrubbers and rotational scrubbers are also possible. Suitable gases are, for example, nitrogen or methane.

The absorption medium stream 20 loaded with C₄-hydrocarbons comprises water. This is separated as stream 22 from the absorption medium in a decanter 21 so as to give a stream 23 which comprises only the water dissolved in the absorption medium.

The absorption medium stream 23 loaded with C₄-hydrocarbons which has been largely freed of water can be heated in a heat exchanger and subsequently introduced as stream 24 into a desorption column 25. In one process variant, the desorption step Dc) is carried out by depressurization and/or heating of the loaded absorption medium. A preferred process variant is utilization of a reboiler at the bottom of the desorption column 25.

The desorption column is preferably a tray column, the wall of which has an explosion-protected design as described above. The desorption column preferably has gastight column trays. The gas-conveying feed lines into the desorption column preferably dip into the liquid. This prevents propagation of the flame front into the column.

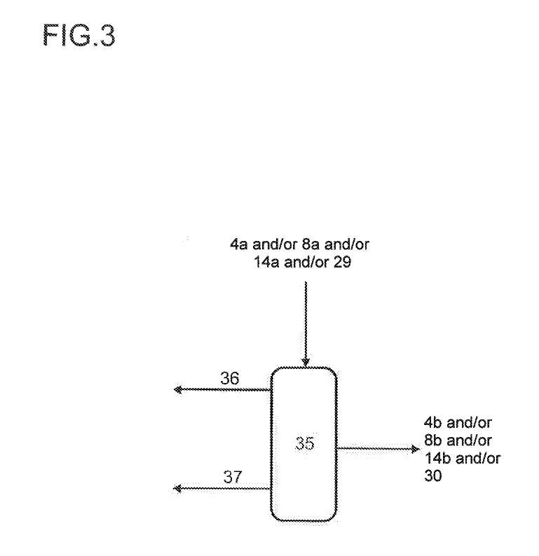

The absorption medium 27 which has been regenerated in the desorption stage can be cooled in a heat exchanger and recirculated as stream 28 to the absorption stage 17. Low boilers such as ethane or propane present in the process gas stream and also high-boiling components such as benzaldehyde, maleic acid and phthalic acid can accumulate in the circulating stream. To limit the accumulation, a purge stream 29 can be taken off and this can either, like streams 14a, 8b and 4b, be separated in a distillation column 35 (FIG. 3) according to the prior art into low boilers 36, regenerated absorbent 30 (FIGS. 2 and 3) and high boilers 37 or preferably be added to streams 14b, 8b or 4b in order to backwash C₄-hydrocarbons dissolved in stream 29 into the process gas stream. If stream 29 is separated in the distillation column 35, the streams 36 and 37 can be burnt and thus utilized to produce energy.

The C₄ product gas stream 31 consisting essentially of n-butane, n-butenes and butadiene generally comprises from 20 to 80% by volume of butadiene, from 0 to 80% by volume of n-butane, from 0 to 10% by volume of 1-butene and from 0 to 50% by volume of 2-butenes, where the total amount is 100% by volume. Furthermore, small amounts of isobutane can be comprised.

Part of the condensed overhead output from the desorption column, which comprises mainly C₄-hydrocarbons, is recirculated as stream 34 to the top of the column in order to increase the separation performance of the column.

The C₄ product gas streams 32 and 33 are subsequently separated by extractive distillation in step E) using a solvent which is selective for butadiene into a stream comprising butadiene and the selective solvent and a stream comprising n-butenes.

The extractive distillation can, for example, be carried out as described in “Erdöl und Kohle—Erdgas—Petrochemie”, volume 34(8), pages 343 to 346, or “Ullmanns Enzyklopädie der Technischen Chemie”, volume 9, 4^{th} edition 1975, pages 1 to 18. For this purpose, the C₄ product gas stream is brought into contact with an extractant, preferably an N-methylpyrrolidone (NMP)/water mixture, in an extraction zone. The extraction zone is generally configured in the form of a scrubbing column comprising trays, random packing elements or ordered packing as internals. This generally has from 30 to 70 theoretical plates so as to achieve a sufficiently good separation action. The scrubbing column preferably has a backwashing zone at the top of the column. This backwashing zone serves to recover the extractant comprised in the gas phase by means of a liquid hydrocarbon runback, for which purpose the overhead fraction is condensed beforehand. The mass ratio of extractant to C₄ product gas stream in the feed to the extraction zone is generally from 10:1 to 20:1. The extractive distillation is preferably carried out at a temperature at the bottom in the range from 100 to 250° C., in particular at a temperature in the range from 110 to 210° C., a temperature at the top in the range from 10 to 100° C., in particular in the range from 20 to 70° C., and a pressure in the range from 1 to 15 bar, in particular in the range from 3 to 8 bar. The extractive distillation column preferably has from 5 to 70 theoretical plates.

The extractive distillation column preferably has an explosion-protected design as described above. The extractive distillation column is preferably a tray column having gastight column trays. The gas-conveying feed lines into the extractive distillation column preferably dip into the liquid.

Suitable extractants are butyrolactone, nitriles such as acetonitrile, propionitrile, methoxypropionitrile, ketones such as acetone, furfural, N-alkyl-substituted lower aliphatic acid amides such as dimethylformamide, diethylformamide, dimethyl-acetamide, diethylacetamide, N-formylmorpholine, N-alkyl-substituted cyclic acid amides (lactams) such as N-alkylpyrrolidones, in particular N-methylpyrrolidone (NMP). In general, alkyl-substituted lower aliphatic acid amides or N-alkyl-substituted cyclic acid amides are used. Dimethylformamide, acetonitrile, furfural and in particular NMP are particularly advantageous.

However, it is also possible to use mixtures of these extractants with one another, e.g. NMP and acetonitrile, mixtures of these extractants with cosolvents and/or tert-butyl ethers, e.g. methyl tert-butyl ether, ethyl tert-butyl ether, propyl tert-butyl ether, n-butyl or isobutyl tert-butyl ether. NMP is particularly useful, preferably in aqueous solution, preferably with from 0 to 20% by weight of water, particularly preferably with from 7 to 10% by weight of water, in particular with 8.3% by weight of water.

The overhead product stream from the extractive distillation column comprises essentially butane and butenes and small amounts of butadiene and is taken off in gaseous or liquid form. In general, the stream consisting essentially of n-butane and 2-butene comprises up to 100% by volume of n-butane, from 0 to 50% by volume of 2-butene and from 0 to 3% by volume of further constituents such as isobutane, isobutene, propane, propene and C₅⁺-hydrocarbons.

The stream consisting essentially of n-butane and 2-butene can be added in its entirety or in part to the C₄ feed to the ODH reactor. Since the butene isomers in this recycle stream consist essentially of 2-butenes and 2-butenes are generally oxidatively dehydrogenated more slowly to butadiene than is 1-butene, this recycle stream can be catalytically isomerized before being fed into the ODH reactor. In this way, the isomer distribution can be set so as to correspond to the isomer distribution present at thermodynamic equilibrium.

In a step F), the stream comprising butadiene and the selective solvent is separated by distillation into a stream consisting essentially of the selective solvent and a stream comprising butadiene.

The stream obtained at the bottom of the extractive distillation column generally comprises the extractant, water, butadiene and small proportions of butenes and butane and is fed to a distillation column. In this, butadiene can be obtained at the top, At the bottom of the distillation column, a stream comprising extractant and possibly water is obtained, with the composition of the stream comprising extractant and water corresponding to the composition as is introduced into the extraction. The stream comprising extractant and water is preferably recirculated to the extractive distillation.

In one variant, a butadiene-comprising extractant is taken off via a side offtake and transferred to a desorption zone in which the butadiene is desorbed from the extractant solution. The desorption zone can, for example, be configured in the form of a scrubbing column which has from 2 to 30, preferably from 5 to 20, theoretical plates and optionally a backwashing zone having, for example, 4 theoretical plates. This backwashing zone serves to recover the extractant comprised in the gas phase by means of a liquid hydrocarbon runback composed of butadiene, for which purpose the overhead fraction is condensed. Ordered packing, trays or random packing elements are provided as internals. The distillation is preferably carried out at a temperature at the bottom in the range from 100 to 300° C., in particular in the range from 150 to 200° C., and a temperature at the top in the range from 0 to 70° C., in particular in the range from 10 to 50° C. The pressure in the distillation column is preferably in the range from 1 to 10 bar. In general, a lower pressure and/or an elevated temperature prevail in the desorption zone compared to the extraction zone.

The desired product stream obtained at the top of the column generally comprises from 90 to 100% by volume of butadiene, from 0 to 10% by volume of 2-butene and from 0 to 10% by volume of n-butane and isobutane. To purify the butadiene further, a further distillation according to the prior art can be carried out.

The wall of the distillation column preferably has an explosion-protected design as described above. The distillation column is preferably a tray column having gastight column trays. The gas-conveying feed lines into the distillation column preferably dip into the liquid in order to prevent propagation of the flame front into the column.

The invention also relates to an apparatus for carrying out the process for preparing butadiene from n-butenes, which comprises:
A) feed line for providing a feed gas stream a comprising n-butenes;
B) feed lines for introducing the feed gas stream a comprising n-butenes and an oxygen-comprising gas into at least one dehydrogenation zone, dehydrogenation zone comprising a dehydrogenation reactor for the oxidative dehydrogenation of n-butenes to butadiene, giving a product gas stream b comprising butadiene, unreacted n-butenes, water vapor, oxygen, low-boiling hydrocarbons, possibly carbon oxides and possibly inert gases;
C) feed line for introducing the product gas stream b into a cooling stage and at least one cooling stage comprising at least one quenching column and at least one compression stage comprising at least one compressor and optionally one or more intermediate coolers between the compressors, where the product gas stream b is brought into contact with at least one circulated coolant to give at least one condensate stream c1 comprising water and a gas stream c2 comprising butadiene, n-butenes, water vapor, oxygen, low-boiling hydrocarbons, possibly carbon oxides and possibly inert gases;
D) facility for separating off incondensable and low-boiling gas constituents comprising oxygen, low-boiling hydrocarbons, possibly carbon oxides and possibly inert gases as gas stream d2 from the gas stream c2 by absorption of the C₄-hydrocarbons comprising butadiene and n-butenes in at least one circulated absorption medium, giving at least one absorption medium stream loaded with C₄-hydrocarbons and the gas stream d2, and facility for subsequent desorption of the C₄-hydrocarbons from the loaded absorption medium stream to give a C₄ product gas stream d1;
E) extractive distillation column for separating the C₄ product stream d1 by extractive distillation using a solvent which is selective for butadiene into a stream e1 comprising butadiene and the selective solvent and a stream e2 comprising n-butenes;
F) distillation column for distillation of the stream e1 comprising butadiene and the selective solvent to give a stream f1 consisting essentially of the selective solvent and a butadiene-comprising stream f2;

wherein one or more of the apparatuses used for carrying out steps A) to F) are configured so as to be explosion-protected, liquid-conveying pipes are configured so as to be explosion-protected and gas conduits are configured so as to be detonation-protected.

In particular, one or more of the following apparatuses are designed so as to be explosion-protected:
the dehydrogenation reactor used in step B);
the quenching column used in step C);
the compressors used in step C);
the intermediate coolers used in step C);
the distillation column used in step F);
the decanter used in the solvent regeneration unit.

The invention is illustrated by the following example.

### EXAMPLE

A commercial plant for producing 1,3-butadiene from n-butenes comprises, inter alia,
reactors for catalytic oxidative dehydrogenation
a quenching column for cooling the product gas stream
compressors for compressing the product gas stream
an absorption column for separating off the inert gas constituents as overhead product from the C4 fraction as bottom product

In order to ensure a satisfactory catalyst operating life, the reactors are operated using an oxygen excess of 6% by volume at the reactor outlet. The streams at the entry into the reactors (n-butenes, air, steam, nitrogen) are equipped with certified quantity measurements and ratio regulation. The condensation of water vapor comprised in the product gas in the quenching column increases the oxygen concentration in the overhead product from the absorption column to above 7% by volume. Apparatuses affected in the plant sections mentioned are designed so as to be explosion-protected and the connecting pipes are designed so as to be detonation-protected. As additional safety element, a certified oxygen measurement is installed in the overhead product from the absorption column, by means of which the oxygen concentration in the system can be monitored continuously. Possible measuring principles for these analytical instruments are laser measurements or paramagnetic measurements or a combination of the two measuring principles.

The safety concept according to the invention allows the design and safe operation of commercial plants for the oxidative dehydrogenation of butenes to butadiene, in particular in processes in which an excess of oxygen at the outlet of the oxidation reactor is necessary.

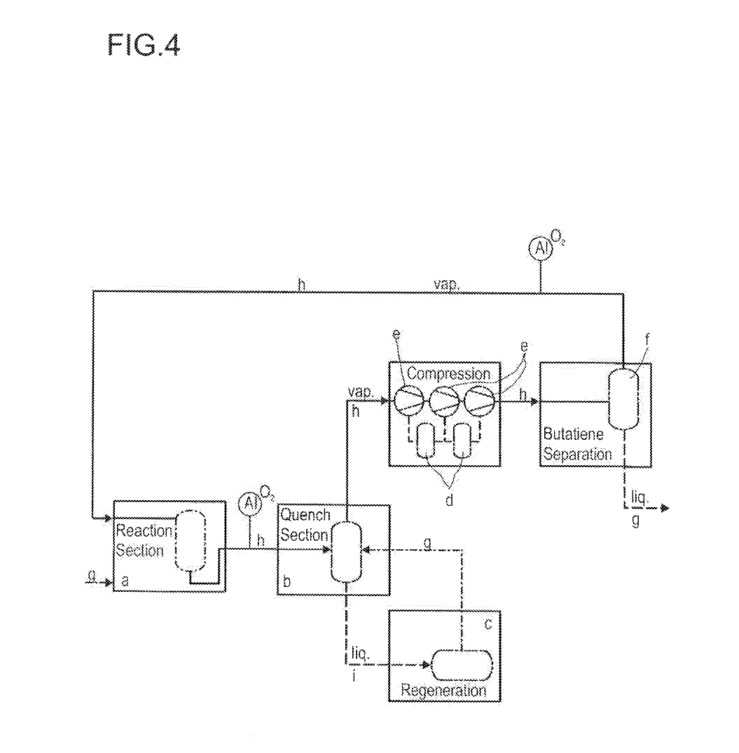

FIG. 4 shows

a) reactor in the reactor section having an explosion-protected design

b) quenching column in the quenching section having an explosion-protected design

c) solvent regeneration unit having an explosion-protected design

d) intermediate cooler using mesitylene as coolant in the compression section as direct cooler having an explosion-protected design

e) compression stages (compressors) in the compression section having an explosion-protected design

f) butadiene column in the butadiene isolation section having an explosion-protected design

g) explosion-protected gas conduits

h) detonation-protected gas conduits

i) explosion-protected liquid conduits

In all column, the gas feed lines dip into the liquid.

All pipes for liquids to and from the columns have an explosion-protected design in order to prevent mechanical overloading of the pipes conveying liquid in the event of an explosion in a vessel.

All gas pipes between the sections have a detonation-protected design.

1.-15. (canceled)

16. A process for preparing butadiene from n-butenes, which comprises the following steps:
A) providing a feed gas stream a comprising n-butenes;
B) introducing the feed gas stream and an oxygen-comprising gas into at least one dehydrogenation zone comprising a dehydrogenation reactor and oxidative dehydrogenation of n-butenes to butadiene, giving a product gas stream b comprising butadiene, unreacted n-butenes, water vapor, oxygen, low-boiling hydrocarbons, optionally carbon oxides and optionally inert gases;
C) cooling and compressing the product gas stream b in at least one cooling stage comprising at least one quenching column and in at least one compression stage comprising at least one compressor and optionally one or more intermediate cooling units between the compressors, with the product gas stream b being brought into contact with at least one circulated coolant to give at least one condensate stream c1 comprising water and a gas stream c2 comprising butadiene, n-butenes, water vapor, oxygen, low-boiling hydrocarbons, possibly carbon oxides and possibly inert gases;
D) separating incondensable and low-boiling gas constituents comprising oxygen, low-boiling hydrocarbons, possibly carbon oxides and possibly inert gases as gas stream d2 from the gas stream c2 by absorption of the C4-hydrocarbons comprising butadiene medium stream loaded with C4-hydrocarbons and the gas stream d2, and subsequent desorption of the C4-hydrocarbons from the loaded absorption medium stream to give a C4 product gas stream d1;
E) separating the C4 product stream d1 by extractive distillation using a solvent which is selective for butadiene into a stream e1 comprising butadiene and the selective solvent and a stream e2 comprising n-butenes;
F) distilling the stream e1 comprising butadiene and the selective solvent to give a stream f1 consisting essentially of the selective solvent and a stream f2 comprising butadiene;
wherein the steps (i) to (iii) below are carried out:
(i) avoidance of formation of explosive gas mixtures by monitoring of the oxygen concentration in the oxygen-comprising gas streams fed into 5 the dehydrogenation zone and control of the mass flows of oxygen comprising gas streams and gas streams comprising hydrocarbons in such a way that no explosive gas mixtures can be formed;
(ii) interruption of the introduction of the oxygen-comprising gas mixture into the dehydrogenation zone when a limit value for the oxygen concentration in the dehydrogenation gas mixture is exceeded;
(iii) carrying out of the steps A) to F) in apparatuses which are configured so as to be explosion-protected, where liquid-conveying pipes are configured so as to be explosion-protected and gas conduits are configured so as to be detonation-protected.

17. The process according to claim 16, wherein one or more of the following apparatuses are designed so as to be explosion-protected:
the dehydrogenation reactor used in step B);
the quenching column used in step C);
the compressors used in step C);
the intermediate coolers used in step C);
the distillation column used in step F); or
the decanter used in the solvent regeneration unit.

18. The process according to claim 16, wherein the intermediate coolers used in step C) are configured as direct coolers.

19. The process according to claim 16, wherein distillation columns used in steps D), E) and/or F) are configured as tray columns having gastight column trays.

20. The process according to claim 16, wherein gas feed lines into the distillation columns used in steps D), E) and/or F) dip into the liquid.

21. The process according to claim 16, wherein gas conduits and gas-conveying apparatuses have such a ratio of length:diameter that detonation is ruled out.

22. The process according to claim 21, wherein the ratio of length:diameter is not more than 50:1.

23. The process according to claim 16, wherein the cooling stage Ca) is carried out in two cooling stages Ca1) and Ca2) and both cooling stages are designed as direct cooling units.

24. The process according to claim 23, wherein at least part of the coolant which has passed through the second cooling stage Ca2) is fed as coolant to the first cooling stage Ca1).

25. The process according to claim 23, wherein the stage Cb) generally comprises at least one compression stage Cba) and at least one cooling stage Cbb), where the gas which has been compressed in the compression stage Cba) is brought into contact with a coolant in the at least one cooling stage Cbb).

26. The process according to claim 25, wherein the coolant of the cooling stage Cbb) is the same organic solvent which is used as coolant in the cooling stage Ca) and at least part of this coolant is, after passing through the at least one cooling stage Cbb), fed as coolant to the cooling stage Ca).

27. The process according to claim 23, wherein the circulated coolant is mesitylene.

28. The process according to claim 16, wherein step D) comprises the steps Da) to Dc):
Da) absorption of the C₄-hydrocarbons comprising butadiene and n-butenes in an absorption medium to give an absorption medium stream loaded with C₄-hydrocarbons and the gas stream d2,
Db) removal of oxygen from the absorption medium stream loaded with C₄-hydrocarbons from step Da) by stripping with an incondensable gas stream and
Dc) desorption of the C₄-hydrocarbons from the loaded absorption medium stream to give a C₄ product gas stream d1.

29. An apparatus for carrying out the process for preparing butadiene from n-butenes according to claim 16, which comprises:
A) feed line for providing a feed gas stream a comprising n-butenes;
B) feed lines for introducing the feed gas stream a comprising n-butenes and an oxygen-comprising gas into at least one dehydrogenation zone, dehydrogenation zone comprising a dehydrogenation reactor for the oxidative dehydrogenation of n-butenes to butadiene, giving a product gas stream b comprising butadiene, unreacted n-butenes, water vapor, oxygen, low-boiling hydrocarbons, possibly carbon oxides and possibly inert gases;
C) feed line for introducing the product gas stream b into a cooling stage and at least one cooling stage comprising at least one quenching column and at least one compression stage comprising at least one compressor and optionally one or more intermediate coolers between the compressors, where the product gas stream b is brought into contact with at least one circulated coolant to give at least one condensate stream c1 comprising water and a gas stream c2 comprising butadiene, n-butenes, water vapor, oxygen, low-boiling hydrocarbons, possibly carbon oxides and possibly inert gases;
D) facility for separating off incondensable and low-boiling gas constituents comprising oxygen, low-boiling hydrocarbons, possibly carbon oxides and possibly inert gases as gas stream d2 from the gas stream c2 by absorption of the C₄-hydrocarbons comprising butadiene and n-butenes in at least one circulated absorption medium, giving at least one absorption medium stream loaded with C₄-hydrocarbons and the gas stream d2, and facility for subsequent desorption of the C₄-hydrocarbons from the loaded absorption medium stream to give a C₄ product gas stream d1;
E) extractive distillation column for separating the C₄ product stream d1 by extractive distillation using a solvent which is selective for butadiene into a stream e1 comprising butadiene and the selective solvent and a stream e2 comprising n-butenes;
F) distillation column for distillation of the stream e1 comprising butadiene and the selective solvent to give a stream f1 consisting essentially of the selective solvent and a butadiene-comprising stream f2;
wherein
one or more of the apparatuses used for carrying out steps A) to F) are configured so as to be explosion-protected, liquid-conveying pipes are configured so as to be explosion-protected and gas conduits are configured so as to be detonation-protected.

30. The apparatus according to claim 29, wherein one or more of the following apparatuses are designed so as to be explosion-protected:
the dehydrogenation reactor used in step B);
the quenching column used in step C);
the compressors used in step C);
the intermediate coolers used in step C);
the distillation column used in step F);
the decanter used in the solvent regeneration unit.
